(19) United States
(12) Patent Application Publication
(10) Pub. No.: US 2014/0316267 A1
(43) Pub. Date: Oct. 23, 2014

(54) NON-INVASIVE ASSESSMENT OF LIVER FAT BY CRAWLING WAVE DISPERSION WITH EMPHASIS ON ATTENUATION

(71) Applicants: Christopher T. Barry, Rochester, NY (US); Deborah J. Rubens, Rochester, NY (US); Kevin J. Parker, Rochester, NY (US)

(72) Inventors: Christopher T. Barry, Rochester, NY (US); Deborah J. Rubens, Rochester, NY (US); Kevin J. Parker, Rochester, NY (US)

(73) Assignee: University of Rochester, Rochester, NY (US)

(21) Appl. No.: 14/239,048

(22) PCT Filed: Aug. 15, 2012
(86) PCT No.: PCT/US12/50934

§ 371 Date: Jun. 17, 2014

Related U.S. Application Data
(60) Provisional application No. 61/523,642, filed on Aug. 15, 2011.

(51) Int. Cl. A61B 8/08; A61B 8/00

(52) U.S. Cl. CPC A61B 8/0858; A61B 8/485; A61B 8/5223; A61B 8/4483; A61B 8/4444
USPC 600/438

(57) ABSTRACT

Using a modified ultrasound device, crawling waves are applied to the liver over a range of shear wave frequencies. Dispersion measurements are obtained that reflect tissue viscosity and these correlate with the degree of steatosis. A device for the process has an actuator on either side of the ultrasound transducer to apply shear waves, which interfere to produce the crawling waves.

### REFERENCE TO RELATED APPLICATIONS

The present application claims the benefit of U.S. Provisional Patent Application No. 61/523,642, filed Aug. 15, 2011. Related subject matter is disclosed in U.S. Provisional Patent Application No. 61/487,025, filed May 17, 2011. The disclosures of the above-cited applications are hereby incorporated by reference in their entireties into the present disclosure.

### STATEMENT OF GOVERNMENT INTEREST

This invention was made with government support under Grant Nos. 5 ROI AG016317 and 5 RO1AG29804 awarded by National Institutes of Health. The government has certain rights in the invention.

### FIELD OF THE INVENTION

The present invention is directed to assessment of liver fat and more particularly to non-invasive assessment of liver fat, e.g., for diagnostic purposes or to track changes over time in response to therapy or progression of disease.

### DESCRIPTION OF RELATED ART

There is growing concern about nonalcoholic fatty liver disease (NAFLD), a major cause of chronic liver disease. The most serious manifestation, nonalcoholic steatohepatitis (NASH), is an increasingly common cause of end-stage liver disease. Although NASH is known to be associated with the metabolic syndrome (obesity, insulin resistance, and hypertriglyceridemia), the natural history of NAFLD progressing to NASH is incompletely understood. Because of the increasing incidence of fatty liver disease and also the important role fat (or “steatosis”) plays in the evaluation of liver donors for transplantation, it is critically important to improve the ability to diagnose the entire spectrum of NAFLD and to understand its pathophysiology. One essential and needed advance is the development of an inexpensive and easy-to-use instrument that could be widely available for researchers to assess the degree of steatosis in the liver, repeatedly, painlessly, and noninvasively.

The gold standard for assessing the degree of hepatic steatosis is biopsy. Although the risk of bleeding post procedure is low and the risk of mortality is estimated to be between 0.01% and 0.1%, biopsy is not always logistically possible (especially in an organ donation setting), and the small amount of tissue procured during biopsy may not reflect the global degree of fatty infiltration. Furthermore, liver biopsies are disliked by patients and are sometimes misinterpreted due to processing artifacts or pathologist's error. Therefore, a reliable noninvasive means of fat determination would be quite beneficial.

Ultrasound is an inexpensive and readily available screening tool for steatosis (as determined by increased diffuse echogenicity due to parenchymal fat inclusions), but the sensitivity ranges from 60-94% and specificity of 66-95% in determining hepatic steatosis. Transient elastography, a technique that measures the velocity of propagation of shear waves through tissue to determine stiffness, has been shown to correlate with histologic stages between 3-5 of liver fibrosis. However, this method cannot measure steatosis when the output is a single “stiffness” estimate. In fact, steatosis confounds shear wave measurements of fibrosis, and this issue is clinically significant given that NASH patients have varying degrees of these two variables. MRI techniques show promise but are in the research stage and would likely be more expensive and time-consuming than ultrasound techniques.

Although other methods exist to estimate steatosis, such as proton magnetic resonance spectroscopy (¹H MRS) and bioimpedence, the former is logistically cumbersome in a clinical setting, and the latter requires probes to be placed into the liver, thereby severely limiting its clinical utility because of safety issues.

### SUMMARY OF THE INVENTION

It is therefore an object of the invention to provide an inexpensive and easy-to-use instrument that can be widely available for researchers to assess the degree of steatosis in the liver.

It is another object of the invention to provide such an instrument that can do so repeatedly, painlessly, and noninvasively.

To achieve the above and other objects, the present invention is built upon a discovery.

We have determined that increasing amounts of fat in the liver will increase the dispersion (that is, the frequency dependence or slope) of the speed of shear waves, while slightly reducing the speed of sound at lower shear wave frequencies. That effect may be the consequence of adding a viscous (and highly lossy) component to the liver, which otherwise would exhibit a strong elastic component with lower dispersion. The addition of microsteatotic fat within hepatocytes results in a macroscopic change in the biomechanical properties of the liver. For example, if the liver is modeled simply as a Voight model, the addition of fat cells adds to the viscosity (dashpot element), and that also increases the dispersion of shear waves propagating in the liver. Furthermore, we have determined that crawling waves, which are an interference pattern of shear waves, can be induced within the liver and imaged by Doppler Ultrasound scanners. The analysis of the crawling wave pattern results in an estimate of the shear wave velocity. When repeated over multiple frequencies from 80 to 300 Hz (or higher in smaller animal livers), the resulting data provide the dispersion estimates that are correlated to steatosis.

The invention is further built upon the following additional discovery. The quantity of fat in the liver can be assessed by measuring the dispersion (or rate of increase with frequency) of the speed of sound and the attenuation of shear waves in the liver. The “crawling waves” methods are the preferred embodiment for applying shear waves and analyzing them for estimation of the dispersion, the key measurement. The inventors believe the attenuation (and its dispersion) may be more sensitive to small changes in the liver fat content than shear wave speed dispersion.

The invention uses the principles of elastography to measure steatosis as distinct from fibrosis.

An ultrasound based approach to measuring steatosis represents a profound advance, as it promises to be safe, cost effective, objective, and expedient. Having such a tool available for animal models, and ultimately for routine clinical use, will have a major impact on the pace of fatty liver disease research and assessment of treatments delivered to patients suffering from the metabolic syndrome.

The present invention allows simultaneous measurements of fat and fibrosis, representing a breakthrough that will be particularly important in the care of patients with NASH. In that population, it is important to gauge progression of fibrosis, and steatosis can confound those measurements. The present invention allows careful separation of the interactions of varying degrees of fat and fibrosis on elastography measurements.

The invention could be applied to humans or animals and for diagnostic purposes or to track changes over time in response to therapy or progression of disease. It has the potential to replace liver biopsies, which are invasive and can have complications and errors.

### BRIEF DESCRIPTION OF THE DRAWINGS

A preferred embodiment of the present invention will be set forth in detail with reference to the drawings, in which:

FIGS. 1a and 1b are plots of the relationship between liver stiffness (shear velocity) and viscosity (dispersion or frequency dependence—vertical axis) in steatotic and lean specimens;

FIG. 2 is a plot of a theoretical pattern of crawling waves excited from surface vibration sources;

FIG. 3 is a plot of an experimental pattern of crawling waves excited from a top surface with two vibration sources;

FIG. 4 is a plot showing a compilation of phantom results for shear velocity;

FIG. 5 is a plot showing a compilation of phantom results for attenuation;

FIG. 6A is an image of H&E staining of a lean mouse liver;

FIG. 6B is an image of H&E staining of an obese mouse liver;

FIG. 6C is an image of oil red O staining of a lean mouse liver;

FIG. 6D is an image of oil red O staining of an obese mouse liver;

FIG. 7 is a plot of compiled mouse liver results;

FIG. 8 is a plot of human liver results; and

FIG. 9 shows a schematic plan for the modified hand-held imaging transducer according to the preferred embodiment.

### DETAILED DESCRIPTION OF THE PREFERRED EMBODIMENT

A preferred embodiment of the present invention will be set forth in detail with respect to the drawings, in which like reference numerals refer to like elements throughout.

The preferred embodiment builds on the principles of elastography to include measurements of dispersion (the frequency dependence of shear waves), which indicates viscosity within the liver. By applying crawling waves to the liver over a range of shear wave frequencies between 80-300Hz, the resulting dispersion measurements (change over frequency) enable the user to separate out the distinct effects of fibrosis (increased stiffness with little dispersion) and fat (softer and more viscous with more dispersion).

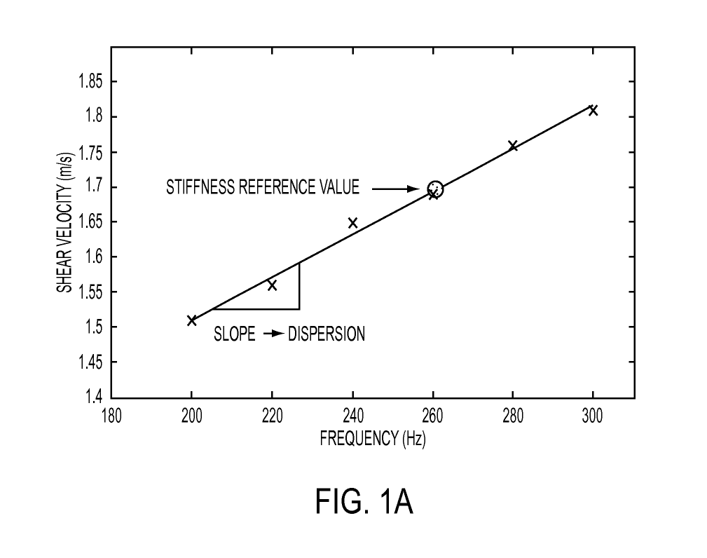

FIGS. 1a and 1b illustrate that separation. FIG. 1a shows a plot of shear velocity in m/s as a function of frequency in Hz. The slop of the line gives the dispersion. A stiffness reference value is shown. FIG. 1b shows two plots of dispersion (slope) as a function of shear velocity. The upper plot shows greater viscosity with increasing fat content. The lower plot shows that with increasing collagen content, the tissue becomes stiffer and more elastic.

The concept of crawling waves was introduced into the elastography field in 2004. Two shear wave sources are placed on the two opposite sides of a sample, driven by sinusoidal signals with slightly offset frequencies. The shear waves from the two sources interact to create interference patterns, which are visualized by the vibration sonoelastography technique. Estimations of local shear velocity can be made from the shear wave propagation pattern and, thus, the shear modulus.

Several approaches have been proposed to estimate local shear velocity from the crawling wave patterns, including a method based on a local spatial frequency estimator (LFE), estimation by moving interference pattern arrival times, and the local autocorrelation method for both 1-D and 2-D shear velocity recoveries. A study of the congruence between the last technique and mechanical measurement validated the imaging modality for quantification of soft tissue properties.

The CrW technique has been used to depict the elastic properties of biological tissues including radiofrequency ablated hepatic lesions in vitro, human skeletal muscle in vitro, and excised human prostate. The preferred embodiment is concerned with crawling waves in the liver.

Crawling waves are interference patterns set in motion by creating a relative frequency shift between the two counter-propagating waves. The discrete version of the detected vibration amplitude square |u|² of the interference of plane shear waves is:

$${\vert u ( m , n , r ) \vert}^{2} = 2 {e}^{- α D} [ cosh ( 2 α {nT}_{n} ) + cos ( 2 {knT}_{n} + Δ {knT}_{n} - Δ k \frac{D}{2} + Δω {rT}_{r} ) ] , \qquad ( 1 )$$

where

α is the attenuation coefficient of the medium, which is a function of frequency and fat content,

D is the separation of the two sources,

ω, the angular frequency measured in radians per second, is 2π times the frequency (in Hz),

k, the wave number measured in radians per meter, is 2π divided by the wavelength λ (in meters), which is a function of frequency and fat content,

Δω is the frequency difference, Δk is the wave number difference between the two waves,

m, n, and r are the spatial vertical index, the spatial lateral (shear wave propagation direction) index, and the time index, respectively, and

Tₙ and Tᵣ are the spatial sampling interval along the lateral direction and the temporal sampling interval, respectively.

By taking the spatial derivative of the phase argument φ of the cosine term of eqn. 1 along the lateral direction, the relationship between local spatial frequency and shear wave velocity is derived for the discrete model:

$$\begin{matrix}{ω}_{spatial} = \frac{\partial φ}{\partial n} \\ = ( 2 k + Δ k ) {T}_{n} \\ = \frac{2 π ( 2 f + Δ f )}{{v}_{shear}} .\end{matrix} \qquad ( 2 )$$

where f is the vibration frequency with the unit of s⁻¹ and vₛₕₑₐᵣ is the local shear wave speed.

vₛₕₑₐᵣ was then calculated based on the relationship:

$${v}_{shear} = \frac{f}{{k}_{spatial}} , \qquad ( 3 )$$

where kₛₚₐₜᵢₐₗ is the spatial frequency with the unit of m⁻¹. In nearly incompressible soft tissues the relationship between shear wave velocity and elastic moduli is

$${v}_{shear} = \sqrt{\frac{E}{3 ρ}} , \qquad ( 4 )$$

where E is Young's modulus, a measure of the stiffness of an isotropic elastic material; and ρ is the density of the medium

There are a number of different ways to calculate the local spatial frequency of a digital signal. One such way involves an autocorrelation technique to estimate the phase derivative of a complex signal sequence.

The phase derivative equals the phase of the autocorrelation R at 1 lag:

$$\frac{\partial φ}{\partial n} = arctan ( \frac{ [ R ( 1 ) ]}{ [ R ( 1 ) ]} ) \qquad ( 5 )$$

The autocorrelation term is calculated by

$$\begin{matrix}R ( 1 ) = \frac{1}{N - 1} {\sum }_{i = n}^{n + N - 2} {s}_{A}^{*} ( i ) {s}_{A} ( i + 1 ) \\ = \frac{1}{N - 1} {\sum }_{i = n}^{n + N - 2} \frac{y ( i ) x ( i - 1 ) - y ( i - 1 ) x ( i )}{x ( i ) x ( i - 1 ) + y ( i ) y ( i - 1 )} ,\end{matrix} \qquad ( 6 )$$

where N is the number of pixels in an estimator kernel, and s_{A} is the analytical signal of |u(m, n, r)|².

Combining Equation (2) and Equation (5), the 1-D shear wave velocity is estimated by

$${〈 {v}_{shear } 〉}_{n} = \frac{2 π ( 2 f + Δ f ) {T}_{n}}{arctan ( \frac{ [ R ( 1 ) ]}{ [ R ( 1 ) ]} )} . \qquad ( 7 )$$

The 2-D shear wave velocity is given by

$${〈 {v}_{shear } 〉}_{2 D} = \frac{{〈 {v}_{shear } 〉}_{m}}{\sqrt{{( \frac{{〈 {v}_{shear } 〉}_{m}}{{〈 {v}_{shear } 〉}_{n}} )}^{2} + 1}} . \qquad ( 8 )$$

In theory, taking the derivative of a phase can provide a very high resolution, but it is very sensitive to noise. Noise reduction is needed before calculating the gradient.

In the preferred embodiment, a hand-held ultrasound transducer is modified to include two parallel vibration sources. The theory for waves produced by a thin beam in contact with the upper surface of a semi-infinite elastic medium was derived by Miller and Pursey in 1954. When the thin bar presses tangentially into the surface of the medium, shear waves are produced in a beam pattern that maximizes at around 45 degrees with respect to the surface. The Miller-Pursey solution has been extended in the preferred embodiment by including two sources and deriving the interference pattern between the two sources as a superposition.

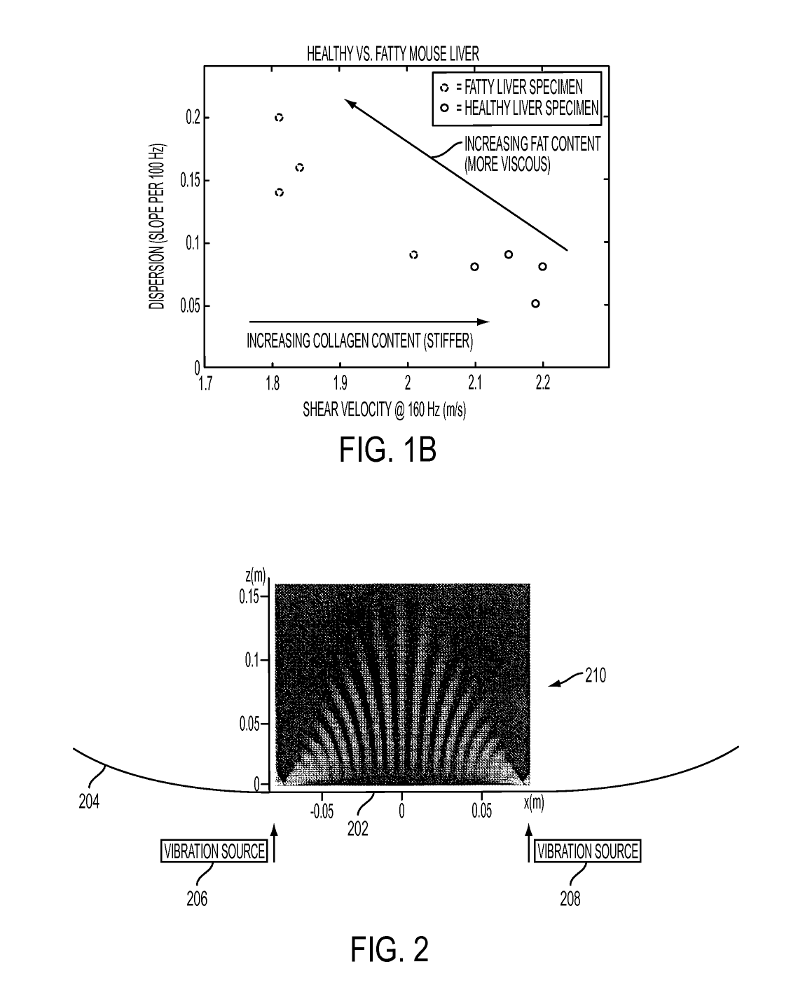

The above will now be described with reference to FIG. 2. Consider a long thin strip 202 placed in close contact with a semi-infinite large, uniform homogeneous elastic solid 204 and vibrating normal to the surface of the medium under the control of two vibration sources (strip loads) 206, 208. The solution for the vibration field in the far field is:

$$\begin{matrix}{u}_{z} = a \cdot {e}^{iπ / 4} \cdot cos θ \cdot \sqrt{\frac{2}{π \cdot R}} \cdot \frac{2 {μ}^{5 / 2} \cdot {sin}^{2} θ \cdot \sqrt{{μ}^{2} \cdot {sin}^{2} θ - 1}}{{F}_{0} ( μ \cdot sin θ )} \cdot {e}^{- iμ R} + \frac{i \cdot cos θ \cdot ( {μ}^{2} - 2 \cdot {sin}^{2} θ )}{{F}_{0} ( sin θ )} \cdot {e}^{- i R} , & ( 9 ) \\ {u}_{x} = a \cdot {e}^{iπ / 4} \cdot cos θ \cdot \sqrt{\frac{2}{π \cdot R}} \cdot \frac{2 {μ}^{5 / 2} \cdot {sin}^{2} θ \cdot \sqrt{{μ}^{2} \cdot {sin}^{2} θ - 1}}{{F}_{0} ( μ \cdot sin θ )} \cdot {e}^{- iμ R} + \frac{i \cdot sin θ \cdot ( {μ}^{2} - 2 \cdot {sin}^{2} θ )}{{F}_{0} ( sin θ )} \cdot {e}^{- i R} , & ( 10 )\end{matrix}$$

where u_{z} is the vibration amplitude in the z (depth) direction, uₓ is the vibration amplitude in the x (transverse) direction, a is the width of the strip load, θ is the angle from the normal direction, and R is the distance from the origin. F₀ is defined as: F₀=(2x²−μ²)²−4x²·\sqrt{(x²⁻1)·(x²−μ²)}; μ=(c₁₁/c₄₄); c₁₁ is the bulk modulus and the c₄₄ is the shear modulus.

The compressional wave is neglected for the following two reasons. First, the wavelength of the compressional wave is typically as long as a few meters, which is not useful in resolving the livers or other structures and cannot be supported in small centimeter sized organs. Second, since the bulk modulus is nearly 1000 times larger than the shear modulus in soft glandular tissue, the amplitude of the compressional wave is actually very small and thus has little contribution to the total pattern.

So, for a normal vibration strip source, the z component and the x component of the shear wave are:

$$\begin{matrix}{u}_{z} = a \cdot {e}^{iπ / 4} \cdot cos θ \cdot \sqrt{\frac{2}{π \cdot R}} \cdot \frac{2 {μ}^{5 / 2} \cdot {sin}^{2} θ \cdot \sqrt{{μ}^{2} \cdot {sin}^{2} θ - 1}}{{F}_{0} ( μ \cdot sin θ )} \cdot {e}^{- iμ R} + \frac{i \cdot cos θ \cdot ( {μ}^{2} - 2 \cdot {sin}^{2} θ )}{{F}_{0} ( sin θ )} \cdot {e}^{- i R} , & ( 11 ) \\ {u}_{x} = a \cdot {e}^{iπ / 4} \cdot cos θ \cdot \sqrt{\frac{2}{π \cdot R}} \cdot \frac{2 {μ}^{5 / 2} \cdot {sin}^{2} θ \cdot \sqrt{{μ}^{2} \cdot {sin}^{2} θ - 1}}{{F}_{0} ( μ \cdot sin θ )} \cdot {e}^{- iμ R} + \frac{i \cdot sin θ \cdot ( {μ}^{2} - 2 \cdot {sin}^{2} θ )}{{F}_{0} ( sin θ )} \cdot {e}^{- i R} , & ( 12 )\end{matrix}$$

Next, a superposition of the vibration field created by two strip loads 206, 208 placed side by side with a separation of a certain distance D will be analyzed. The left branch of the right strip load and the right branch of the left strip load interfere with each other and localize the energy into a region 210. That region can be imaged with a Doppler ultrasound scanner.

The beam pattern of the double-strip load is related to the wavelength of the propagating shear waves. In theory that provides an experimental method to measure the shear wave velocity in the material. The shear modulus can be further obtained from those interference patterns, by the estimators given above. We note that the use of the local estimators is restricted to a zone near the proximal surface, since at some depth the interference patterns become weak and also exhibit geometrical spreading.

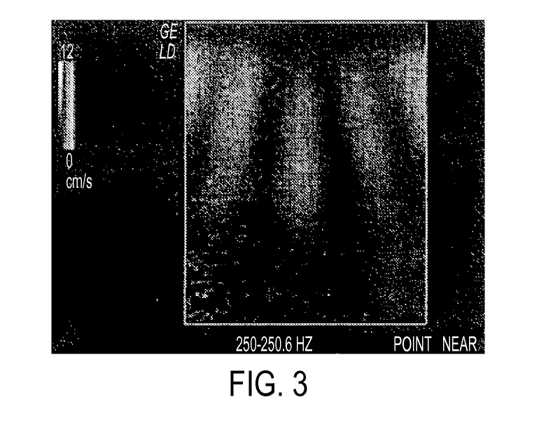

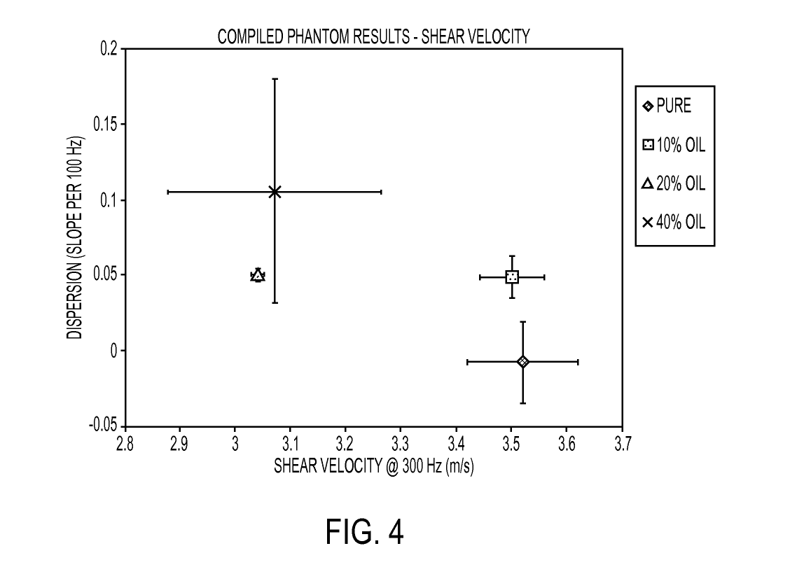

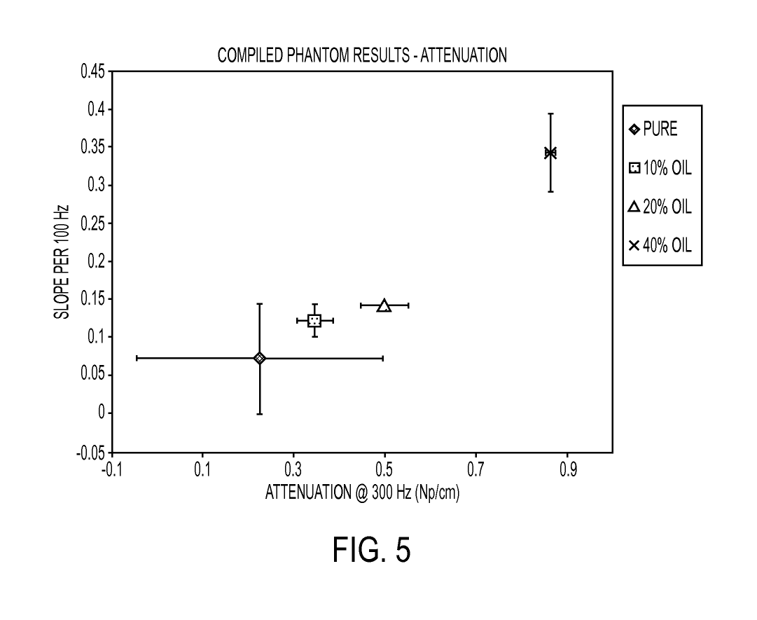

An experimental result of crawling waves in a phantom is given in FIG. 3. FIG. 4 shows compiled phantom results for shear velocity. Dispersion (slope per 100 Hz) is plotted against shear velocity at 300 Hz in m/s for pure, 10% oil, 20% oil, and 40% oil phantoms. FIG. 5 shows compiled phantom results for attenuation. Dispersion is plotted against attenuation at 300 Hz in Np/cm for the same phantoms. FIG. 5 shows an approximately linear relation between dispersion and attenuation.

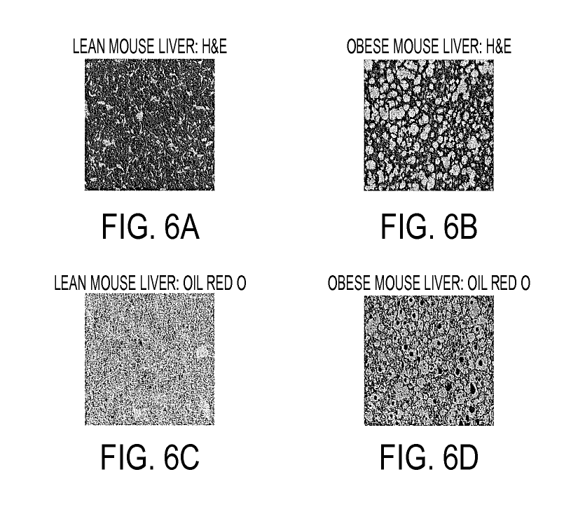

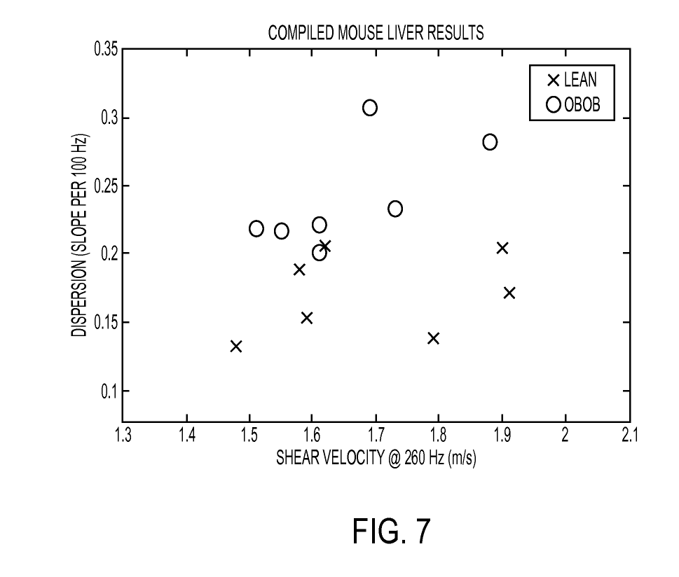

To model the effect of steatosis, the inventors found that in comparing fatty castor oil slurries with pure gelatin slurries, dispersion is higher (0.1 m/s per 100 Hz) and shear velocity is lower (2.95 m/s) in the fatty slurry relative to the normal slurry (0.019 m/s per 100 Hz and 3.8 m/s, respectively). To further test the relationship, twenty mouse liver specimens (10 lean ob/+ fed a regular diet and 10 steatotic ob/ob fed a high fat diet) were embedded in two 8% gelatin (300 Bloom Pork Gelatin, Gelatin Innovations Inc., Schiller Park, Ill., USA) cube-shaped molds after a hepatectomy. The mold was placed in an ice water bath for approximately 90 minutes, cooling from a temperature of roughly 50° Celsius to 15° Celsius. The solid gelatin phantoms were removed from their respective molds and allowed to rest at room temperature for 10 minutes prior to scanning Scanning was performed as described below, but with a non-portable (bulky) set of vibration sources suitable for benchtop experiments. In ob/ob mice the mean dispersion slope was 0.15+/−0.015 m/s per 100 Hz, compared to lean mice at 0.075+/−0.02 m/s per 100 Hz. The average shear velocity was 1.87+/−0.10 m/s at 160 Hz in ob/ob mice and 2.16+/−0.05 m/s at 160 Hz in lean mice (see FIG. 1). Histologic analysis of H&E sections and Oil Red O staining confirms the absence of steatosis in the lean mice and approximately 65% steatosis in the ob/ob mice (FIGS. 6A-6D show representative samples). FIG. 7 shows compiled mouse liver results for dispersion plotted against shear velocity.

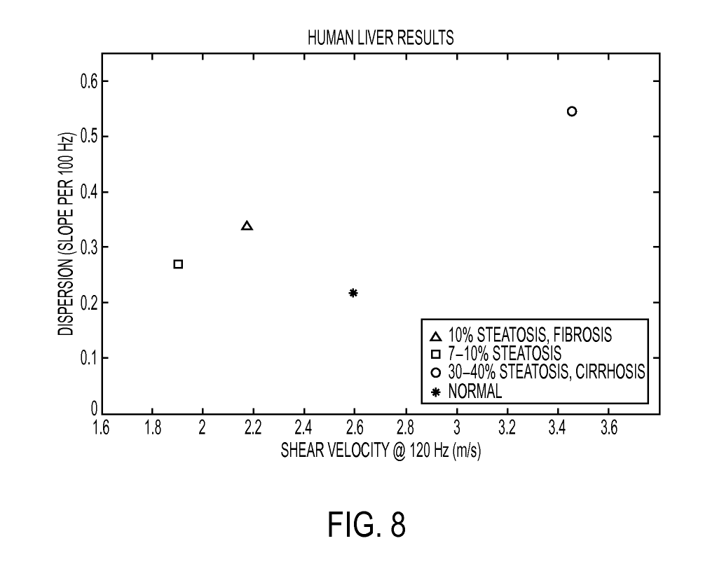

Finally, in human liver tissue, measurements from a patient with 40% macrosteatosis and grade 3 fibrosis on histological exam showed a dispersion slope of 0.68 m/s per 100 Hz and shear velocity of 2.5 m/s compared to a normal liver specimen with a dispersion slope of 0.01 m/s per 100 Hz and shear velocity of 2.08 m/s. In this case, the shear velocity is higher in the patient with macrosteatosis presumably because of the increased degree of fibrosis compared to the normal liver. These results lend strong support to our hypothesis and demonstrate that we have all of the technical skills in place to perform our proposed experiments. FIG. 8 shows human liver results for dispersion plotted against shear velocity.

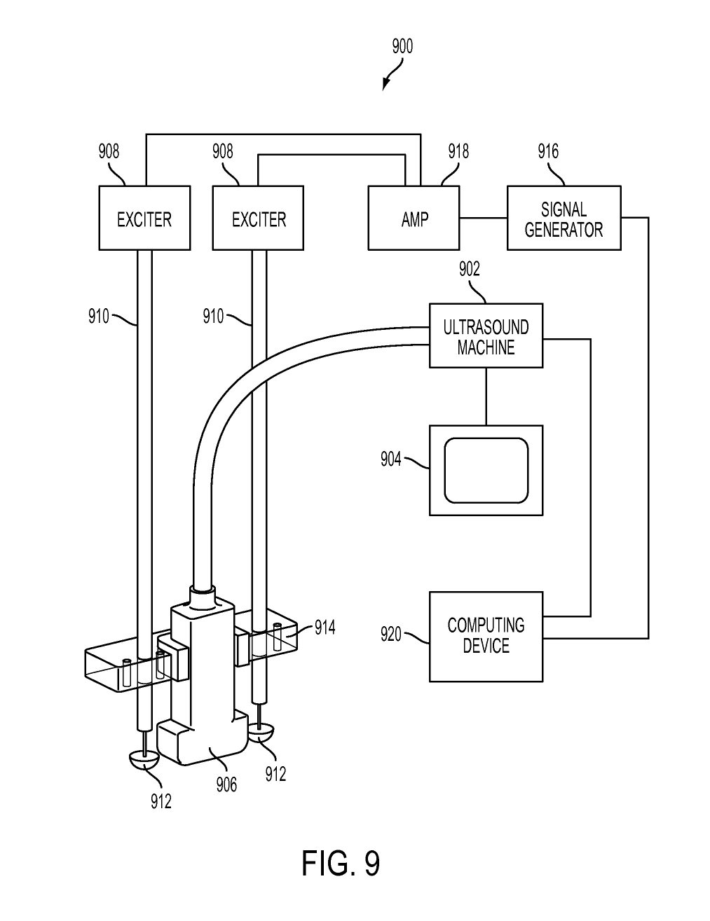

An example of a system 900 according to the preferred embodiment will now be described with reference to FIG. 9. A GE Logic 9 ultrasound machine 902 (GE Healthcare, Milwaukee, Wis., USA) is modified to show vibrational sonoelastographic images in the color-flow mode on its display or other output 904. An ultrasound transducer 906 (M12L, GE Healthcare, Milwaukee, Wis., USA) will be connected to the ultrasound machine and placed on top of the region of interest. It is a linear array probe with band width of 5-13 MHz.

Two piston vibration exciters 908 (Model 2706, Briiel & Kjaer, Naerum, Denmark) will be employed to generate the needed vibrations between approximately 80 and 300 Hz. These sources are too bulky to attach to the transducer 906, so precision aircraft-style flexible cables 910 will be employed to conduct the vibrations towards the surface, The cables 910 and contacts 912 are attached by a frame 914 on each side to the 15 MHz imaging transducer 906 (in the center). This imaging transducer 906 images a region of interest up to 4 cm in width, and the attached cables 910 (which provide the vibration at the surface and therefore create the crawling wave pattern within the field of view of the imaging transducer) are connected in such a way that the entire apparatus can be hand-held and easily placed into position. At the tips of the cables 910 are rubber contacts 912 for firm but comfortable transmission of the vibration. Displacements of less than 700 microns peak to peak at the source are sufficient because the Doppler imaging is capable of resolving shear wave displacements in the range of 2-10 microns within deep tissue. The shear wave signals are generated by a two-channel signal generator 916 (Model AFG320, Tektronix, Beaverton, Oreg., USA) and amplified equally by a power amplifier 918 (Model 5530, AE Techron, Elkhart, Ind., USA), which is connected to the pistons. The interference pattern of the shear waves produces “Crawling Waves” which are readily imaged by Doppler techniques.

A computing device included in, or in communication with, the signal generator 916 or the ultrasound machine 902 or both can perform all necessary computations. As an illustrative example, FIG. 9 shows a computing device 920 in communication with both the ultrasound machine 902 and the signal generator 916.

The vibrational sources will be driven at frequencies offset by 0.35 Hz, creating a moving interference pattern in the imaging plane termed a crawling wave (CrW). A region of interest (ROI) is selected from each of the sonoelastographic images of CrW propagation through the embedded liver specimens, and a projection of the wave image over the axis perpendicular to the interference pattern is fit to a model. From the model parameters, a wavelength value is derived and hence, a shear velocity of the liver medium can be calculated. Sonoelastographic images gathered from frequencies generated between 80-400 Hz provide an outline of the frequency-based dispersion of shear velocity estimates.

The present invention builds the foundation for assessing fatty liver and related diseases in a painless and noninvasive way that will also be affordable. It will lessen the need for the unpleasant liver biopsy and also provide researchers who study animal models a convenient way of tracking the progress of new treatments. It can be used routinely to assess patients who have NASH, NAFLD, and metabolic syndrome. It can be used to gauge the efficacy of dietary and lifestyle modifications and other treatments.

As we gain experience with a larger number of patients, there will be a database of results correlated against pathology and biopsy results. This will enable us to compare a measurement of shear wave speed, shear wave attenuation, and their dispersions, against the database and thereby give a likely grade to the patient, e.g., “This measurement suggests liver fat content of 30%.” The database could be stored in the processor, which refers to the database to determine a “grade” or likely fat content for a particular patient and measurement.

While a preferred embodiment has been set forth in detail above, those skilled in the art who have reviewed the present disclosure will readily appreciate that other embodiments can be realized within the scope of the invention. For example, specific brand names and model numbers are illustrative rather than limiting, as are specific frequency ranges and other numerical values. Also, more than two vibration sources can be used. Therefore, the present invention should be construed as limited only by the appended claims.

We claim:

1. A method for non-invasive assessment of fat in a liver of a patient, the method comprising:
(a) applying shear waves to the liver from a plurality of locations to cause the shear waves to interfere in the liver, the shear waves optionally having a frequency offset to create crawling waves in the liver;
(b) repeating step (a) over a plurality of frequencies of the shear waves;
(c) during steps (a) and (b), detecting the interfering waves using a transducer;
(d) analyzing the interfering waves detected in step (c) in a processor to determine a dispersion of at least one of a speed and an attenuation of the shear waves; and
(e) from the dispersion determined in step (d), assessing the fat in the liver.

2. The method of claim 1, wherein the transducer comprises an ultrasound transducer.

3. The method of claim 1, wherein step (a) comprises applying the shear waves as counter-propagating shear waves from two of said locations.

4. The method of claim 3, wherein the transducer is located between said two locations.

5. The method of claim 1, wherein the plurality of frequencies comprise frequencies within a range of 80 to 400 Hz.

6. A probe for non-invasive assessment of fat in a liver of a patient, the probe comprising:
a plurality of actuators for applying shear waves to the liver from a plurality of locations to cause the shear waves to interfere in the liver, the shear waves optionally having a frequency offset to create crawling waves in the liver; and
a transducer for detecting the interfering waves and for outputting a signal representing the crawling waves to a processor.

7. The probe of claim 6, wherein the transducer comprises an ultrasound transducer.

8. The probe of claim 6, wherein the transducer is disposed between two of said actuators.

9. The method of claim 1, further comprising:
(f) storing a result of step (e) in a database; and
(g) making the database available to the processor for future assessments.

10. A system for non-invasive assessment of fat in a liver of a patient, the system comprising:
a plurality of actuators for applying shear waves to the liver from a plurality of locations to cause the shear waves to interfere in the liver, the shear waves having a frequency offset to create interfering waves in the liver;
a signal generator for controlling the plurality of actuators to apply the shear waves over a plurality of frequencies of the shear waves;
a transducer for detecting the crawling waves and for outputting a signal representing the interfering waves;
a processor, connected to the transducer to receive the signal, for analyzing the interfering waves to determine a dispersion of at least one of a speed and an attenuation of the shear waves; and
an output for outputting a result of analysis from the processor.

11. The system of claim 10, wherein the transducer comprises an ultrasound transducer.

12. The system of claim 10, wherein the actuators are configured to apply the shear waves as counter-propagating shear waves from two of said locations.

13. The system of claim 12, wherein the transducer is located between said two locations.

14. The system of claim 10, wherein the signal generator is configured such that the plurality of frequencies comprise frequencies within a range of 80 to 400 Hz.

15. The system of claim 10, further comprising a database for storing the result of analysis, wherein the database is available to the processor for future assessments.

16. A method for non-invasive assessment of fat in a liver of a patient, the method comprising:
(a) applying shear waves to the liver from a plurality of locations to cause the shear waves to interfere in the liver, the shear waves optionally having a frequency offset to create crawling waves in the liver;
(b) repeating step (a) over a plurality of frequencies of the shear waves;
(c) during steps (a) and (b), detecting the interfering waves using a transducer;
(d) analyzing the interfering waves detected in step (c) in a processor to determine a dispersion of at least one of a speed and an attenuation of the shear waves; and
(e) comparing the dispersion determined in step (d) to a database of results to make an estimate of a percentage of fat in the liver; and
(f) outputting the estimate.
